(19) United States
(12) Patent Application Publication
(10) Pub. No.: US 2024/0304302 A1
(43) Pub. Date: Sep. 12, 2024

(54) ARRHYTHMIA ASSESSMENT MACHINE LEARNING

(71) Applicant: The Vektor Group Inc., San Diego, CA (US)

(72) Inventors: Christopher J. T. Villongco, Oakland, CA (US); Robert Joseph Krummen, Bellevue, WA (US); Joseph Thomas Braidwood, Issaquah, WA (US); Christian David Marton, Jersey City, NJ (US); Douglas Steven Komen, Encinitas, CA (US); Maurice J. Pirio, Seattle, WA (US)

(21) Appl. No.: 18/597,796

(22) Filed: Mar. 6, 2024

Related U.S. Application Data
(60) Provisional application No. 63/450,593, filed on Mar. 7, 2023.

(51) Int. Cl. G16H 20/40; A61B 34/10; G16H 10/60; G16H 15/00; G16H 20/10; G16H 50/30; G16H 50/70

(52) U.S. Cl. CPC G16H 20/40; A61B 34/10; G16H 10/60; G16H 15/00; G16H 20/10; G16H 50/30; G16H 50/70; A61B 2034/104

(57) ABSTRACT

A system is provided that employs a machine learning (ML) decision tree to provide a treatment analysis for an arrhythmia. The ML decision tree includes decision nodes (non-leaf nodes) and treatment analysis nodes (leaf nodes). Each decision node corresponds to a feature derived from electronic health records and has branches corresponding to feature values. A treatment analysis node corresponds to a treatment analysis based on feature values of a path from the root node to that treatment analysis node. To provide a treatment analysis for a candidate, the system identifies a path from the root node to a treatment analysis node based on a candidate feature vector derived from an electronic health record of the candidate and outputs the treatment analysis of the treatment analysis node of the identified path.

### CROSS-REFERENCE TO RELATED APPLICATION(S)

This application claims the benefit of U.S. Pro. App. No. 63/450,593 filed on Mar. 7, 2023 and entitled “ARRHYTHMIA ASSESSMENT MACHINE LEARNING,” which is hereby incorporated by reference it its entirety.

### BACKGROUND

In general, a candidate for an arrhythmia treatment may have limited information to help inform their decision on treatment options. Often, a candidate may rely on anecdotal stories of positive treatment outcomes and negative treatment outcomes of non-journal articles published on the web and experiences of family and friends. The candidate, however, may have had very little in common with the patients with those outcomes. For example, the candidate and the patients may have very different characteristics such as ages, sexes, comorbidities, severity of disease, genetics, and so on. A candidate who decides on a treatment option (e.g., anti-arrythmia drugs, ablation, cardioversion) based on such anecdotal stores may end up having a worse outcome such as a lower quality of life and a shorter life expectancy (which may be characterized as survival or mortality) than if the candidate had decided on a different treatment option (e.g., ablation treatment for an arrhythmia rather than medication). In addition, the decision becomes even more complex when the candidate has available many treatment options and combinations of those treatment options. For example, the treatment options may include an invasive procedure (e.g., an ablation), medication, a wait-and-see approach, and so on. The treatment options themselves have sub-options. For example, an ablation treatment for an atrial fibrillation (AF) may have the sub-options of one or more ablation targets (e.g., pulmonary vein isolation (PVI) and/or an AF source location), various ablation patterns, various medications (e.g., a beta blocker or calcium channel blocker), and so on.

In contrast to the typical candidates, medical providers may rely on the standard of care when recommending a treatment for a certain arrhythmia. The standard of care may be based on accepted medical practices and guidelines established by medical boards, professional societies, and health organizations based on current medical research, technologies, and practices. The standard of care, however, provides general guidelines and is not patient specific. The guidelines are not patient specific in the sense that they are not based on the unique characteristics of each patient such as medical history, demographics, patient wishes, anatomical, electrophysiological, and so on. Moreover, it is difficult for the standard of care to reflect and care providers to consider all the findings of the latest medical research and accompanying statistics to help provide a recommended treatment. For example, the standard of care, for a patient with a certain type of arrhythmia and with a certain comorbidity, may be an ablation at a certain target location. However, a recent journal article or a recent presentation at a conference may suggest that an ablation for such a patient would be more effective with a different target location and/or a secondary target location. Every year hundreds of journal articles and presentations are published that relate to analysis of arrhythmias and their treatments. It is currently challenging for a practicing electrophysiologist to review and analyze all these journal articles to assess the results, the bases, and the merits of the findings and then customize a recommended course of treatment for a candidate based on those findings.

Rather than relying solely on the standard of care and on a medical provider's knowledge of and assessments of recent publications, it would be desirable to have a computer system that would provide medical providers and candidates with information on possible treatments for an arrhythmia and likely outcomes to better inform their decisions. With such information, a medical provider may recommend and/or a candidate may select a treatment that is different from the standard of care. For example, a first treatment option may have mean life expectancy of 5 years, but a median life expectancy of only 2 years. A second treatment option may have a mean life expectancy of only 4 years, but a median life expectancy of 3 years. Although the standard of care and/or a recent publication may indicate the first treatment option, if provided with the second treatment option and the life expectancy information, a candidate might select the second treatment option with a higher chance of living at least 3 years. Moreover, it would be desirable to have a computer system that would provide treatment options and treatment assessments that are tailored to candidate characteristics based on analysis of patients with similar characteristics.

### BRIEF DESCRIPTION OF THE DRAWINGS

FIG. 1 is a block diagram that illustrates components of the AAML system in some embodiments.

FIG. 2 is a flow diagram that illustrates the processing of a generate ML decision tree component of the AAML system in some embodiments.

FIG. 3 is a flow diagram that illustrates the processing of a create node component of the AAML system in some embodiments.

FIG. 4 is a flow diagram that illustrates the processing of a generate treatment analysis component of the AAML system in some embodiments.

FIG. 5 is a flow diagram that illustrates the processing of a walk decision tree component of the AAML system in some embodiments.

FIG. 6 illustrates an example TAML decision tree relating to atrial fibrillation.

### DETAILED DESCRIPTION

An arrhythmia assessment machine learning (AAML) system provides candidate-specific treatment analyses that may be treatment options or treatment assessments. The treatment options and treatment assessments may help inform decisions relating to treatment of arrhythmias. A treatment assessment may be used by medical providers or candidates for treatment when selecting a treatment option, for example, based on a risk/benefit analysis of treatment options factoring in the treatment assessments for those treatment options. A treatment assessment for a treatment option may indicate, for example, cohort success rate, cohort lifespan extension (e.g., from treatment to death), cohort need for a repeat ablation, cohort mortality or survival, and so on. For example, a candidate with an atrial fibrillation (AF) and heart failure with a source location in one part of the left atrium may have the treatment options of an ablation at a target location (and with a certain ablation pattern) or specific medication. The treatment assessments may indicate that, within the candidate's cohort, an ablation has a success rate of 81% and that medication has success rate of 25%. The treatment assessments may also indicate the typical impact on life expectancy such as 5+ years for an ablation and 2+ years for that medication. The treatment assessments may also indicate the impact of no treatment such as the risk of stroke, the life expectancy, and so on. In addition to providing treatment options and treatment assessments, the AAML system may also generate diagnostic assessments such as the presence of an arrhythmia and its source location.

The AAML system is an improvement over traditional computer systems that assist in treating a patient. Such computer systems may provide treatment options only based on a standard of care or on data reported in publications and may not provide treatment assessments. Thus, those treatment options (and any treatment assessments) are not candidate specific. In addition, because the AAML system is computationally efficient, it may be employed to provide treatment options and treatment assessments in real-time. For example, during an ablation procedure a medical provider may acquire new information about the candidate such as an ablation performed at one target location not resulting in termination of the arrhythmia or a new scar tissue being identified. In such a case, the AAML system may be employed to provide treatment options and treatment assessments based on that new information.

To provide candidate-specific treatment options and treatment assessments, the AAML system collects candidate characteristics. The candidate characteristics may be collected from the electronic health records (EHRs) of the candidate or provided via a user interface. As discussed below in more detail, the AAML system extracts relevant features of the candidate characteristics and may derive additional features. An additional feature may be a source location of an arrhythmia derived from an electrocardiogram (ECG) or cardiac anatomical characteristics derived from a computed tomography (CT) scan. The AAML system then employs a treatment option machine learning (TOML) model generated by the AAML system and a treatment assessment machine learning (TAML) model generated by the AAML system to provide treatment options and their treatment assessments. The AAML system may employ different machine learning (ML) architectures for the ML models such as a neural network for the TOML model and a decision tree for the TAML model. Rather than having separate machine learning (ML) models for treatment options and treatment assessments, the AA system may provide a combined treatment option and assessment machine learning (TOAML) model.

In some embodiments, the AAML system may employ an ML model that is an ML decision for the TOML model and the TAML model. A TOML decision tree includes non-leaf nodes (also referred to as decision nodes) relating to features (e.g., candidate characteristics), leaf nodes that each relates to one or more treatment options, and branches (also referred to as links) relating to feature values of the feature on which a decision is made. A TAML decision tree includes non-leaf nodes relating to candidate characteristics and treatment options, leaf nodes relating to treatment assessments, and branches relating to feature values. A feature may be, for example, candidate age, and the feature values may be the ranges of less than 40, 40 to 60, and over 60. Other examples of features may be blood pressure with feature values of low, normal, high, and very high and a heart rate with feature values of bradycardia, normal, and tachycardia.

To provide a treatment option for a candidate, the AAML system generates a candidate feature vector of features with feature values representing data derived from a candidate's EHRs (or input via a user interface) and treatment options for a TAML decision tree. To identify a treatment option based on a candidate feature vector, the AAML identifies a path from the root node of the TOML decision tree to a leaf node that is consistent with the feature values of the candidate feature vector. The treatment option associated with that leaf node is provided as a treatment option for the candidate. To identify a treatment assessment for a treatment option, the AAML identifies a path from the root node of the TAML decision tree to a leaf node. The identified path is consistent with feature values of the candidate feature vector with a treatment option feature with that treatment option as the feature value. Alternatively, if a combined TOAML decision tree is employed, the feature vector would typically not include a treatment option, and each leaf-node may indicate one or more treatment options and their treatment assessments. Some leaf nodes of an ML decision tree may indicate, for example, that the training data used in training the ML decision tree was not sufficient to provide a treatment option or a treatment assessment.

Although the ML models are described primarily in the context of treatments for an arrhythmia, the AML system may be employed to provide treatment options and treatment assessments for other medical conditions such as coronary artery bypass grafts, cancers, neurological disorders, and so on. The AAML system may also employ separate TOML decision trees and TAML decision trees for different cardiac conditions (e.g., AF or premature ventricular contraction (PVC)) and separate TAML decision trees for different treatment options (e.g., pulmonary vein isolation (PVI) and medication). An advantage of an ML decision tree over some other non-decision tree ML models is that it is easier to explain the factors that went into identifying a treatment option or a treatment assessment. Those factors are based on the path from the root node of a ML decision tree to a leaf node.

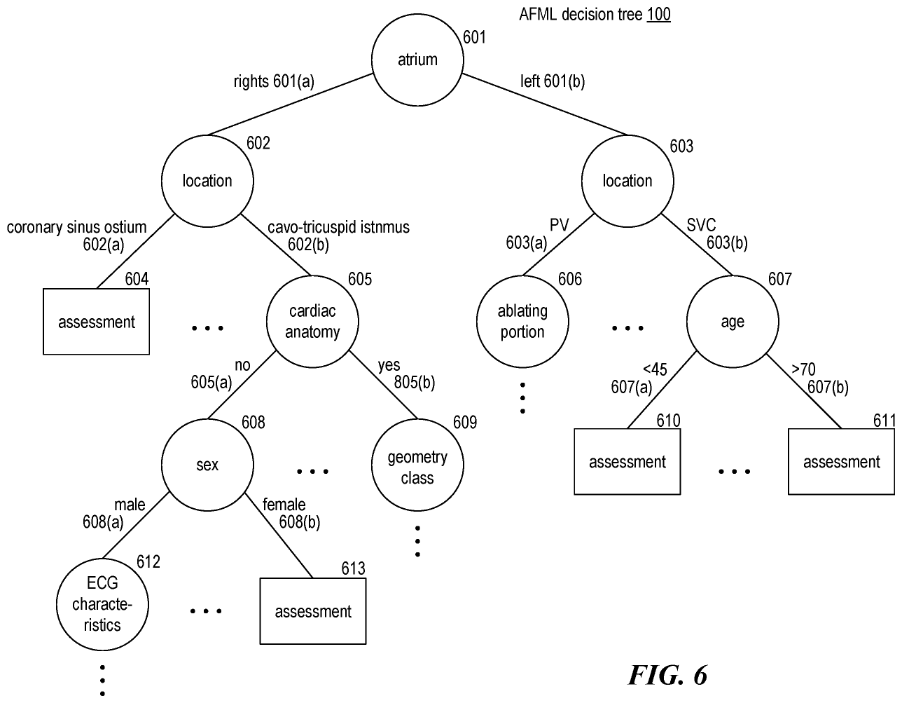

FIG. 6 illustrates an example TAML decision tree relating to atrial fibrillation. The TAML decision tree 600 provides treatment assessments for an ablation treatment option for an atrial fibrillation. The TAML decision tree includes nodes 601-613. The circular nodes represent decision nodes (non-leaf nodes), and the rectangular nodes represent treatment assessment nodes (leaf nodes). Nodes 601, 602, 603, and 606 represent treatment options. For example, node 601 (the root node) represents the atrium to be treated, and node 602 represents the target location for an ablation. The remainder of the decision nodes represent candidate features. For example, node 607 represents an age feature, and node 608 represents a sex feature. The branches 601(a)-608(b) represent feature values for features of their parent nodes. For example, branch 601(a) represents the right atrium, branch 602(a) represents the coronary sinus ostium, and branch 608(a) represents the male sex.

To illustrate use of a TOML decision tree and a TAML decision tree consider a candidate who is a male age 72, who has a certain cardiac anatomy characteristic, and who has AF in the left atrium. The AAML system employs the TOML decision tree to identify a treatment option for the candidate that is an ablation based on the source location of the AF in the superior vena cava (SVC), which is not actually in the left atrium. The AAML system then employs the TAML decision tree 600 to identify the treatment assessment for that treatment option. To identify the treatment assessment for the candidate, the AAML system generates a candidate feature vector for the candidate. The treatment option features may include an atrium feature that can have the value of right or left, a location feature that can have a value such as pulmonary vein or cavo-tricuspid isthmus, and ablation pattern feature. The candidate features may include sex, age, and whether the candidate has a certain cardiac anatomical characteristic. If the feature value for a feature is not known, the AAML system may set the feature value to a default value such as age of 50 in the unlikely event that the candidate's age is not known. Also, the AAML system may set the feature value to indicate that it is not available. In such a case, each decision node may have a branch corresponding to the feature value of not available so that a treatment assessment can be identified even when the feature value is not available, and such as treatment assessment would not factor in that feature.

For this candidate, since the decision node 601 is based on the atrium affected by the AF and the candidate's AF is in the left atrium, branch 601(b) is selected which leads to decision node 603. Since decision node 603 is based on source location of the AF and the candidate's location is the superior vena cava, branch 603(b) is selected leading to decision node 607. Since decision node 607 is based on age and the candidate is 72, branch 607(b) is selected which leads to treatment assessment 611. The path from the root node to the treatment assessment node for the candidate would include decision nodes 601, 603, and 607 and treatment assessment node 611. The AAML system then outputs the treatment assessment indicated by treatment assessment node 611. The AAML system may also output an indication of the path to help with the explainability of the treatment assessment. For example, the path would indicate that age is a factor in the treatment assessment, but sex was not.

As another example, consider another candidate who is female age 60, who does not have a certain cardiac anatomical characteristic, and who has an AF in the right atrium. The AAML system employs a TOML decision tree to identify a treatment option or a treatment option may be specified by a medical provider. For example, the treatment option may be an ablation targeting the cavo-tricuspid isthmus. The AAML system then generates a feature vector for the candidate for use in identifying the treatment assessment. The AAML system identifies the path of the TAML decision tree that includes decision nodes 601, 602, 605, and 608 and treatment assessment node 613. The AAML system then outputs the treatment assessment of treatment assessment node 613.

The TAML decision tree 600 is provided as a very simple example to illustrate the structure and use of a TAML decision tree. The example patient features may not actually be relevant to a treatment assessment for a certain treatment option or other patient features may be more relevant. For example, prior ablation history may be more relevant than age for treating an arrhythmia originating in the superior vena cava (SVC). The ellipses indicate that other nodes are not illustrated. For example, the locations associated with the right atrium may also include SVC, crista terminalis, tricuspid valve annulus, Eustachian ridge and valve, right atrial appendage, and atrial septum. The locations for the left atrium may also include left atrial posterior wall, left atrial appendage, mitral valve annulus, ligament of Marshall, left atrial septum, Bachman bundle, and coronary sinus.

Although TAML decision tree 600 includes treatment option nodes, the AAML system may support TAML decision trees that have only candidate features. In such a case, the AAML system may create a decision tree for each treatment option. For example, the AAML system may create a TAML decision tree for a treatment option of an ablation targeting the cavo-tricuspid isthmus and a TAML decision tree for the treatment option of a certain medication. The TAML decision tree for the treatment option of an ablation targeting the cavo-tricuspid isthmus may correspond to the subtree with decision node 605 as its root node.

The AAML system may provide a variety of treatment options that may be different from the standard of care treatment option. For example, the standard of care for certain types of AF may be a pulmonary vein isolation (PVI). The AAML system may use the TOML decision tree to identify a treatment option that is an ablation targeting a different location or that is a certain medication. Since the TOML decision tree may be trained (also referred to as generated) using training data derived from EHRs, the features of the decision nodes may be different from features pertinent to the standard of care. As a result, the identified treatment option for some candidates is likely to be different from the treatment option of the standard of care.

The AAML system may employ the TAML decision tree to identify treatment options for candidates. For example, after generating a feature vector for a candidate, the AAML system may identify paths from the root node to a treatment assessment node that include different treatment option nodes. For example, a TAML decision tree may have a root node with a branch for an ablation pattern and a branch for medicine. The ablation branch may lead to a decision node of ablation pattern. If a medical provider wants treatment assessments for different ablation patterns, the TAML decision tree may be used to identify paths that each include a different ablation pattern. The treatment assessment node of each path provides the treatment assessment for that ablation pattern. Those treatment assessments can be used to help inform which ablation pattern to use.

The AAML system may provide a treatment assessment that indicates within a cohort a PVI may not be particularly effective. In such a case, the AAML system may provide treatment assessments for other treatment options that have been more effective. The AAML system may provide treatment assessments based on the likelihood of a recurrence for treatment options such as ablations targeting different source locations, different antiarrhythmic drug options (e.g., amiodarone), or a cardioversion. If the effectiveness of such a treatment option is indicated as being low for the candidate given the candidate feature vector, the AAML system may suggest that additional information, such as cardiac anatomical information, would be useful in providing a more informed treatment assessment. In such a case, the AAML system may suggest collecting a computed tomography (CT) scan of the candidate from which the cardiac anatomical information can be derived.

The AAML system may also provide treatment option information (derived from the candidate's cohort) such as ablation patterns that have been effective or recurrence locations that may help inform a decision relating to an ablation pattern or an ancillary target location. For example, the cohort members who had an ablation at the mid-posterior LV may have had statistically significant recurrence at the right ventricular outflow tract (RVOT) posterior wall. In such a case, the treatment option information may indicate that the RVOT should be an ancillary target location. The AAML system may also provide information of characteristics of a candidate that may affect the treatment outcome. For example, a candidate with a non-ischemic cardiomyopathy may have a significantly greater chance of an unsuccessful outcome than someone without such a cardiomyopathy. Although the decision may be to proceed with the ablation knowing the chances of an unsuccessful outcome, the decision may alternatively be to first treat with an antiarrhythmic drug or to defer the treatment decision to see how the arrhythmia progresses.

When using a non-decision tree ML model (e.g., neural network), the AAML system may generate a candidate feature vector with candidate features relevant to a decision for a treatment option. The AAML system derives feature values for the candidate features from data extracted from the candidate's EHR. The AAML system inputs the candidate feature vector into an ML model that was trained to provide treatment assessments for that treatment option based on candidate features. Such an ML model may apply weights and biases (learned when training the ML model using training data) to the candidate feature vector to generate a treatment assessment. The candidate feature vector may also include treatment options features. As an example, a candidate feature vector may include the features discussed above in reference to the TAML decision tree 600. The factors that influence a treatment assessment identified using a non-decision tree TAML model may be more difficult to explain than those that influence one identified by a TAML decision tree. Those factors for a TAML decision tree (or TOML decision tree) are based on the path from the root node to a treatment assessment node (or treatment option node).

The AAML system may train an ML model (decision tree or non-decision tree) to generate a treatment assessment for a target treatment option for a target medical condition using training data derived from the EHRs of patients who had the target medical condition and who were treated (or not) with one or more of available treatment options. The treatment assessments may include, for example, likelihood of reducing symptoms, reducing mortality, increasing life expectancy, improving quality of life, and so on. To generate the training data, the AAML system scans the EHRs to identify patients who had the target medical condition (e.g., AF or certain neurological disorder (e.g., epilepsy)). For each patient, the AAML generates a training feature vector with features derived from the EHR of that patient. If a treatment assessment is to provide the life expectancy after a PVI ablation to treat an AF, the features may include arrhythmic electrocardiograms (ECGs) taken prior to the PVI ablation, patient demographic data (e.g., age, sex, and ethnicity), patient medical readings (e.g., blood pressure, heart rate, coronary calcium score, and HDL level) prior to the PVI ablation, comorbidities (e.g., cardiomyopathy), prior treatment history (e.g., a prior ablation location and date of treatment), medical history after a treatment (e.g., arthrosclerosis and date of death), the patient's family history, a CT scan of the patient's heart, a quality of life assessment before and after the PVI ablation (e.g., extracted from EHR comments using machine learning), a medical provider's assessment of the effectiveness of the treatment, catheter path used in an ablation treatment, and so on. A catheter path may specify, for example, entry via the inferior vena cava and through the tricuspid valve. Another feature that may affect a catheter path may be a blockage in a coronary artery which can impact an ablation targeting the epicardium. A TOML model (i.e., decision tree or non-decision tree) may generate a treatment option that includes catheter path. Additional techniques for identifying a catheter path are described in U.S. Pro. App. No. 63/550,020 entitled “Cardiac Catheter Path Planning System” and filed on Feb. 5, 2024, which is hereby incorporated by reference.

For an ML decision tree, the AAML system may generate, for each training feature vector for a patient, one or more labels based on the outcome for the patient. For example, if the treatment option is an AF ablation, the outcomes may indicate the post-treatment lifespan for dead patients and living patients, a quality-of-life assessment, the need for subsequent ablation, and so on. After generating an ML decision tree, the AAML system may generate a treatment assessment for each treatment assessment node based on the labels of the patient feature vectors with feature values that match the path from the root node to that treatment assessment node. For example, the AAML system may generate statistics on life expectancy such as mean and median based on the lifespan of dead patient and/or current lifespan of living patient.

The AAML system may train a separate TAML model (i.e., decision tree or non-decision tree) for each medical condition and for each treatment option in which case the training data would have a label(s) relating to that medical condition and treatment option (e.g., ablation, antiarrhythmic drug, or no treatment). The medical condition of a candidate feature vector is used to select a TAML model. The AAML system may train separate TAML models for a medical condition for patients who were treated and for patient who were not treated.

When a candidate wants an assessment relating to, for example, an ablation treatment for an arrhythmia, the candidate (or medical provider) may collect an ECG via an ECG collection device such as a smartwatch, a Holter monitor, a clinical ECG device, and so on. Upon receiving the ECG, the AAML system may identify the arrhythmia type and available treatment options based on analysis of the ECG. The AAML system may access the candidate's EHR to extract data needed to generate a candidate feature vector for input to a TAML model. If the EHR is not available or all the data for one or more features is not in the EHR, the AAML system may collect from the candidate or medical provider answers to questions so that the feature values for those features can be determined. The AAML system may train separate TAML models for candidates who EHRs are available and for candidates whose EHRs are not available. The TAML model for candidates whose EHRs is not available may be trained with limited features that most candidates would be expected to know.

In some embodiments, a treatment assessment may indicate additional information that would be helpful in providing a more refined treatment assessment or may indicate an alternative treatment option. For example, referring to the TAML decision tree 600, the AAML system may indicate that a more refined treatment assessment could be made if a CT scan indicating the cardiac anatomy is collected. If an initial treatment assessment is based on treatment assessment node 613, the AAML system may identify that the cardiac anatomy feature has no feature value available. In such a case, the AAML system may suggest collecting a cardiac CT scan from which a feature value may be generated. The AAML system may also provide information on the treatment assessment that could be made if the CT scan were collected. For example, the AAML system may identify paths from branch 605(b), which would be taken if the cardiac anatomical information were available, to treatment assessment nodes. The AAML system provides the treatment assessments of those treatment assessment nodes to help inform a decision of whether to collect a CT scan.

The AAML system may identify alternative treatment options that may have a more favorable treatment assessment. For example, the AAML system may determine that a treatment option that is an ablation targeting a PV with a certain ablation pattern has a less favorable treatment assessment than one with a different ablation pattern. For example, the recurrence rates may be 25% and 10%, respectively. The treatment assessment relating to an ablation pattern corresponds to branches from an ablation pattern decision node. The leaf node of each path from that such a branch provides a treatment assessment relating to that ablation pattern.

The AAML system may assist in identifying the type of arrhythmia and the source location (or origin) of the arrhythmia. To identify the type of arrhythmia, the AAML system may employ various techniques that identify the type based on analysis of an arrhythmia ECG such as those described in PCT App. No. PCT/US23/72854 entitled “Automatic Fibrillation Classification and Identification Fibrillation Epoch” and filed on Aug. 24, 2023, which is hereby incorporated by reference. To identify a source location, the AAML system may employ a mapping system that inputs an arrhythmia ECG of the candidate and outputs a source location. Techniques for a mapping system are described in U.S. Pat. No. 10,860,754 entitled “Calibration of Simulated Cardiograms” and issued on Dec. 8, 2020, which is hereby incorporated by reference. The mapping system may identify one source location (monomorphic arrhythmia) or multiple source locations (polymorphic arrhythmia). The mapping system may identify a source location based on a single cycle (or epoch). For each of multiple cycles of an arrhythmic ECG, the AAML system may employ the mapping system to identify a source location associated with that cycle. The source locations that are near each other may be considered to be a cluster that tends to indicate a source location with a high confidence. The AAML system may employ a feature that indicates the number of source locations, a feature for each source location, and so on.

The AAML system may also generate additional implicit features that are not explicit features in an EHR. For example, the AAML system may generate a feature that is a rating of closeness of a target location to a critical structure. If the critical structure is an SA node, the AAML system may identify the location of the SA node (e.g., based on analysis of a CT scan) and calculate the distance between the SA node and the target location. That distance may be used as a feature that would indicate an increased risk of an adverse outcome as the distance decreases. If data is not available to generate a certain feature, the AAML system may set the feature to a substitute value such as an average value, a standard value (e.g., 120/80 for blood pressure), or null value (e.g., to indicate that a value for that feature is not available or that its accuracy is in doubt).

In some embodiments, the AAML system may employ simulations to generate additional training feature vectors and/or to identify treatment options to augment the training feature vectors generated from patient EHRs. For example, the AAML system may run simulations of electrical activity of a patient's heart with cardiac characteristics derived from the patient's EHR. Each simulation assumes a different source location such as in the cavo-tricuspid isthmus or in the coronary sinus ostium. Each simulation is run until the arrhythmia stabilizes. The treatment options may be to perform an ablation targeting one or more target locations. For each treatment option, the AAML system continues the simulation assuming that treatment option to assess the effectiveness of the treatment option. The AAML system may generate a simulated training feature vector based on the patient's EHR augmented with a treatment option of a simulation (e.g., target location) and an indication of whether that treatment option was successful (e.g., arrhythmia terminated). The AAML system may also run simulations on various ablation patterns to determine whether an ablation pattern based on a target location would be successful and augment the training feature vectors based on the results of the simulation. The AAML system may also generate simulated training feature vectors based on modifications to features derived from patient EHR such as running simulations that each adds different scar tissue characteristics to patient cardiac characteristics. Techniques for running such simulations are described in U.S. Pat. No. 11,806,080 entitled “Identify Ablation Patterns for Use in an Ablation” and issued on Nov. 7, 2023, which is hereby incorporated by reference.

Because the AAML system employs machine learning, the AAML system is able to identify features as being relevant to a treatment assessment when the standard of care does not consider those features. For example, assuming that a standard of care would not consider coronary calcium in making a treatment assessment, the AAML system may identify that coronary calcium should be considered. Alternatively, if a standard of care would consider coronary calcium, the AAML system may indicate that coronary calcium should be a more important factor. Coronary calcium may be helpful in providing an assessment relating to a source location on the epicardium or a source location of a ventricular tachycardia (VT). If a candidate has significant calcium in coronary arteries, then the chances of an ablation targeting the epicardium being successful may be relatively low. If the candidate has significant calcium in the aorta, then the chances of an ablation, targeting a source location in the ventricle, being successful may be higher if the treatment option is to access the ventricle via the mitral valve rather than the aorta.

In some embodiments, the AAML system may employ various ML techniques in providing the assessment. The ML techniques may include an ML decision tree, K-Nearest Neighbor (KNN) ML model, a neural network model for classification, and so on.

For an ML decision tree, the AAML system may employ an entropy score to select the feature to be associated with each decision node. The entropy score for a possible feature for a decision node may be based on the distribution of its feature values in node feature vectors for that decision node. A node feature vector for a node has the feature values of the branches along the path from the root node to that node. For example, if a first possible feature for a node has an equal number of node feature vectors for each feature value, the entropy for the first possible feature is considered to be high. In contrast, if a second possible feature has node feature vectors for which 75% have the same feature value, then the entropy for the second possible feature is considered to be low. In such a case, the AAML system would select the second possible feature for that node. The AAML system may also analyze features with continuous values to identify cut points that tend to minimize entropy. For example, as mentioned above, heart rate may be classified as bradycardia (<60), normal (60-100), or tachycardia (>100). However, since heart rate is a continuous value, the AAML system may employ techniques to identify cut points (other than 60 and 100) that would reduce the entropy. For example, a single cut point of 80 or cut points of 50 and 90 may tend to minimize entropy. Techniques for identifying cut points are described in Fayyad, U. M. and Irani, K. B., 1992. On the handling in decision tree of continuous-valued attributes generation. Machine Learning, 8, pp. 87-102., which is hereby incorporated by reference.

The AAML system may employ a depth-first, recursive algorithm to build an ML decision tree. The AAML system may employ a path termination criterion to determine when to terminate a path. The path termination criterion may be, for example, when the percentage of node feature vectors that are associated with the same outcome is above a threshold percentage. For example, if 80% of the node feature vectors have the outcome of success with a PVI, the AAML system may add a treatment assessment node that indicates a treatment assessment of an 80% success rate for a PVI and indicates an average lifespan extension based on those node feature vectors. Other termination criteria may be that the number of node feature vectors is below a threshold number or that the path has reached a maximum depth. In such cases, the AAML system may add a leaf node indicating that a treatment assessment cannot be provided or has a low confidence.

The AAML system may also factor in study data (e.g., statistical data) derived from published studies relating to the treatment options. Many studies have been conducted that evaluate the effectiveness of an ablation location given arrhythmia type and patient characteristics. For example, one study indicates that an ablation at a source location for an AF may be more effective than a PVI or that ablations at both locations may be particularly effective. Various patient characteristics such as sleep apnea, hypertension, or obesity may impact the effectiveness of the treatment. This data is referred to as “training” data because the data can be considered to be used to train an ML model. For example, a treatment assessment may be augmented to include information derived from these studies. Also, features of an EHR may be included in or excluded from a feature vector based on a manual determination of the relevance of a feature to a treatment assessment. For example, ethnicity may not be relevant in a treatment assessment relating to an AF. Example studies are described in Lee, D., Hoffmayer, K. S., Hsu, J. C., Schricker, A., Birgersdotter-Green, U., Raissi, F., Feld, G. K. and Krummen, D. E., 2019. Long-term mode and timing of premature ventricular complex recurrence following successful catheter ablation. Journal of Interventional Cardiac Electrophysiology, 55, pp. 153-160 and Narayan, S. M., Baykaner, T., Clopton, P., Schricker, A., Lalani, G. G., Krummen, D. E., Shivkumar, K. and Miller, J. M., 2014. Ablation of rotor and focal sources reduces late recurrence of atrial fibrillation compared with trigger ablation alone: extended follow-up of the CONFIRM trial (Conventional Ablation for Atrial Fibrillation With or Without Focal Impulse and Rotor Modulation). Journal of the American College of Cardiology, 63(17), pp. 1761-1768, which are hereby incorporated by reference.

In some embodiments, the AAML system may employ a K-Nearest Neighbor (KNN) model to provide an assessment. The training data may be training feature vectors as described above and with labels indicating a treatment assessment. A KNN model may be used without a training phase that is without learning weights or other parameters to represent the training data. In such a case, the candidate feature vector is compared to the training feature vectors to identify a number (e.g., represented by the “K” in KNN) of similar training feature vectors. Once the number of similar training feature vectors are identified, the AAML system analyzes the labels associated with the similar training feature vectors to provide a treatment assessment for the candidate. The AAML system may weigh the treatment assessments of the training feature vectors that are more similar to the candidate feature vector higher than those that are less similar. For example, if there are 10 training feature vectors with four that are very similar and six that are less similar, the AAML system may assign similarity weights of 0.9 to the very similar training feature vectors and 0.2 to the less similar. If three of the four and one of the six have the same treatment outcomes, then the treatment assessment for the candidate is primarily based on that treatment outcome even though most of the 10 have different treatment outcomes. Conceptually, training feature vectors that are very similar are closer to the candidate feature vector in a multi-dimensional space of features, and a similarity weight is based on distance between the feature vectors. The AAML system may employ various techniques to calculate a similarity metric indicating similarity between a candidate feature vector and a training feature vector such as dot product, cosine similarity, Pearson correlation, and so on.

If the number of training feature vectors is large, the AAML system may use various techniques to effectively “compress” the training data during a training phase. For example, the AAML system may employ a clustering technique to identify clusters of training feature vectors that are similar and have the same label. The AAML system may then generate a training feature vector for each cluster (e.g., one from the cluster or one based on mean values for the features) as a cluster feature vector and assign a cluster weight to it based on number of training feature vectors in the cluster. The AAML system would provide an assessment as described above but using the cluster feature vectors and factoring in their cluster weights.

In some embodiments, the AAML system may employ an ML clustering technique (e.g., K-means clustering) to identify clusters of training feature vectors that are similar. For each cluster, the AAML system may generate a treatment assessment based on the labels (outcomes) of the training feature vectors in that cluster. For example, if the medical condition is a PVC and 90% of the labels indicate that an ablation location at the left ventricle outflow tract (LVOT)/coronary cusp was successful, then a 90% success rate may be the treatment assessment for that cluster. To identify the treatment assessment for a candidate, the AAML system may employ a similarity criterion to identify a cluster or clusters based on similarity to the candidate feature vector and provide a treatment assessment based the treatment assessment of those clusters. The similarity criterion may be, for example, a similarity score (e.g., cosine similarity) is above a threshold value, is the highest similarity score, is at least 20% higher than all other similarity scores, is a certain number of standard deviations above the mean similarity score, and so on or a combination of criteria.

In some embodiments, the AAML system may employ a neural network (NN) model to provide a treatment option or a treatment assessment. The AAML system may employ an ML decision tree to generate a treatment assessment and a treatment assessment for each training feature vector. The AAML system may generate training feature vectors labeled with treatment options or treatment assessments. Alternatively, the AAML system may label each training feature vector for a treatment assessment with one or more outcomes (e.g., recurrence and lifespan extension). In such a case, the output of the TAML model may be a probability of an outcome.

The AAML system may perform a feature selection analysis to identify features that are relevant to a treatment option or treatment assessment. For example, features can be selected that have a high mutual information with the treatment assessment (e.g., based on Fano's inequality). Also, linear regression may be used to calculate weights for features, and the features with weights near zero may be deemed irrelevant to the treatment option or the treatment assessment. The relevant features could then be used to train an ML model.

The AAML system may filter out patients whose information may not be informative to a treatment assessment. For example, if the cause of death of a patient is not related to an arrhythmia (e.g., car accident), the AAML system might not use the patient feature vector of that patient when training an ML model to make a life expectancy assessment but may use it when training a ML model to make a quality-of-life assessment. The AAML system may also filter out patient feature vectors for which the mapping source location indicated by the mapping system does not match the ground truth source location of a successful ablation as indicated by the EHRs. The reason for filtering out such a patient feature vector is that an ablation performed at a location other than the predicted source location may not be informative in assessing an outcome of an ablation performed at the predicted source location. The AAML system may train a separate filter ML model using all the features except for the source location feature and with labels indicating whether a predicted source location matches the ground truth source location. The filter ML model can be applied to a candidate feature vector to determine whether there would be a match. If there is a match, the AAML system may then apply another ML model that makes an assessment based on the predicted source location. The filter ML model effectively identifies candidates for which an assessment made by the AAML system may have a relatively low confidence.

In some embodiments, once an ML model is trained, the AAML system may generate graphs to assist in a treatment assessment. A graph may illustrate, for example, life expectancy (y-axis) based on patient age (x-axis). To generate such a graph, the AAML system may generate graph feature vectors with feature values selected based on the distribution of feature values in the training feature vectors except for an age feature. For each graph feature vector, the AAML system inputs the graph feature vector into the ML model multiple times with different ages such as ages 25 through 100. The output may be considered the life expectancy for a candidate of that age based on a certain treatment option. The AAM system may calculate the life expectancy for a given age by calculating an average of all the life expectancies based on graph feature vectors with that age.

Since the computational expense of applying an ML model to a candidate feature vector is relatively low, the AAML system may allow a medical provider to select the feature values for a graph feature vector. In which case, the graph would indicate the life expectancy for patients with those specific features. The AAML system may also generate graphs on demand that are customized to a candidate. For example, the value for an unknown feature (e.g., non-ischemic cardiomyopathy) may not be known for a candidate. The AAML system may generate a graph with values for that unknown feature (x-axis) and life expectancy (y-axis). To populate the graph, the AAML system applies the ML model to, for each possible value of that unknown feature, a candidate feature vector with the unknown feature is set to that possible value. The AAML populates the graph based on the output treatment assessments.

The AAML system may also provide a user interface that allows for the exploration of an ML decision tree. The AAML system may display portions of the ML decision tree such a subtree that includes and identifies the path from the root node to a treatment assessment for a candidate feature vector. A medical provider may scroll through the decision tree to better understand features and feature values that may affect the treatment assessment such as availability of a CT scan.

The AA system may scan EHRs to extract information for the training data using de-identification techniques as required by the relevant jurisdiction (e.g., HIPAA or GDPR). Alternatively, the AA system may access extracted information that has already been de-identified.

For some assessments, the number of features that are relevant to that assessment may be large. In such a case, the AAML system may employ a dimensionality technique (e.g., principal component analysis) to reduce the dimension of a feature vector. Such a reduction in dimensionality may be appropriate for some techniques such as KNN and a NN but not for other techniques such as an ML decision tree.

The AAML system may provide a candidate portal through which a candidate can access treatment assessments that are personalized to their medical history. The candidate portal may be provided via a client application running on client device (e.g., smartphone, smartwatch, or tablet) or via a web interface. The candidate portal provides a user interface through which a candidate can review and correct relevant data (e.g., a feature of the candidate feature vector) retrieved from their EHR or can enter relevant data that is not available via an EHR (e.g., if their EHR is not accessible). The candidate portal may interface with an ECG device (e.g., smartwatch) to collect a candidate ECG in real time when the patient is requesting a treatment assessment for a treatment option (e.g., certain medication) that the candidate specifies.

When collecting data for identifying a treatment option or treatment assessment, the candidate portal may determine the features for which values are needed dynamically. If an ML decision tree is used to identify the treatment option or treatment assessment, the data requested is based on the path of the ML decision tree from the root node to a certain decision node. For example, if the path leads to a decision node for sex, the feature of the decision node to which the female branch connects may be obesity and to which the male branch connects may be ischemic events. In such a case, a female would be asked to enter a weight, and a male would be asked to enter heart attack information.

The AAML system may also provide a recommended course of treatment for a candidate. For example, for each possible treatment option, the AAML system may initially apply a TAML model to a candidate feature vector to identify a treatment assessment for each treatment option. The AAML system may then, for each treatment option, augment the candidate feature vector to indicate an outcome (e.g., negative) for that treatment option. The AAML system may then apply a TAML model to each augmented candidate feature vector to identify a treatment assessment for each treatment option assuming the outcome of the prior treatment. This process can be repeated multiple times to generate a tree of treatment options and outcomes for different courses of treatment. For example, a medical provider may identify four treatment options for candidate. To assist in recommending a course of treatment, a TAML model may be employed to generate a treatment assessment for each of the four treatment options. For each treatment option, the TAML model may then be employed to generate, for each other treatment option, a treatment assessment with a feature value based on that treatment option. The AAML system then continues for each possible sequence of treatment options. If there are four treatment options, then there are 24 possible courses of treatment. The recommended course of treatment for a candidate may also be adjusted after each treatment.

In addition to the ML models mentioned above, the AAML system may employ any of a variety or combination of supervised, semi-supervised, self-supervised, unsupervised, or reinforcement learning ML models including a neural network such as fully connected, convolutional, recurrent, or autoencoder neural network, or restricted Boltzmann machine, a support vector machine, a Bayesian classifier, decision tree, generative adversarial networks, transformers, and so on. When the ML model is a deep neural network, the ML model is trained using training data that includes features and feature values derived from data and labels corresponding to the data. For example, the EHR data may include images of ECGs with a feature being the image itself, and the labels may be a characteristic indicated by the ECGs (e.g., QRS integral). The training may result in a set of weights and biases for activation functions of the layers of the deep neural network. The trained deep neural network can then be applied to a candidate feature vector to generate a label for that candidate feature vector. When the ML model is a support vector machine, a hyper-surface is found to divide the space of possible inputs candidate feature vectors. For example, the hyper-surface attempts to split the positive examples (e.g., patient with long lifespan extension) from the negative examples (e.g., patient with a short lifespan extension) by maximizing the distance between the nearest of the positive and negative examples to the hyper-surface. The trained support vector machine can then be applied to as candidate feature vector to generate a treatment assessment for the candidate. An ML model may generate values of discrete domain (e.g., classification), probabilities, and/or values of a continuous domain (e.g., regression value, classification probability).

Various techniques can be used to train a support vector machine such as adaptive boosting, which is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates increasingly on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, loss function, and search algorithm. The architecture defines the functional form relating the inputs to the outputs (in terms of network topology, unit connectivity, and activation functions). The search in weight space for a set of weights that minimizes the loss function is the training process. A neural network model may use a radial basis function (RBF) network and a standard or stochastic gradient descent as the search technique with backpropagation.

A convolutional neural network (CNN) has multiple layers such as a convolutional layer, a rectified linear unit (ReLU) layer, a pooling layer, a fully connected (FC) layer, and so on. Some more complex CNNs may have multiple convolutional layers, pooling layers, and FC layers. Each layer includes a neuron for each output of the layer. A neuron inputs outputs of prior layers (or original input) and applies an activation function to the inputs to generate an output.

A convolutional layer may include multiple filters (also referred to as kernels or activation functions). A filter inputs a convolutional window, for example, of an image, applies weights to each pixel of the convolutional window, and outputs value for that convolutional window. For example, if the static image is 256 by 256 pixels, the convolutional window may be 8 by 8 pixels. The filter may apply a different weight to each of the 64 pixels in a convolutional window to generate the value.

An activation function has a weight for each input and generates an output by combining the inputs based on the weights. The activation function may be a rectified linear unit (ReLU) that sums the values of each input times its weight to generate a weighted value and outputs max(0,weighted value) to ensure that the output is not negative. The weights of the activation functions are learned when training a ML model. The ReLU function of max(0,weighted value) may be represented as a separate ReLU layer with a neuron for each output of the prior layer that inputs that output and applies the ReLU function to generate a corresponding “rectified output.”

A pooling layer may be used to reduce the size of the outputs of the prior layer by downsampling the outputs. For example, each neuron of a pooling layer may input 16 outputs of the prior layer and generate one output resulting in a 16-to-1 reduction in outputs.

An FC layer includes neurons that each input all the outputs of the prior layer and generate a weighted combination of those inputs. For example, if the penultimate layer generates 256 outputs and the FC layer inputs a neuron for each of three classifications (e.g., three treatment assessments), each neuron inputs the 256 outputs and applies weights to generate value for its classification.

One example of a CNN is a U-Net ML model. The U-Net ML model includes a contracting path and an expansive path. The contracting path includes a series of max pooling layers to reduce spatial information of the input image and increase feature information. The expansive path includes a series of upsampling layers to convert the feature information to the output image. The input and output of a U-Net represent an image such as an image of patient ECG as input and various ECG characteristics (e.g., QRS integral) as output.

Multimodal ML combines different modalities of input data to make a prediction. The modalities may be, for example, images (e.g., CT scans), text (e.g., textual comments), and audio (e.g., recordings) of an EHR.

In one multimodal ML approach, referred to as “early fusion,” data of the different modalities is combined at the input stage and is then trained on the multimodal data. The training data for these modalities include a collection of sets of an image, related text, and related audio and labels. The image, text, and audio may be used in its original form or preprocessed, for example, to reduce its dimensionality by compressing the data into byte arrays or applying a principal component analysis. Also, the resolutions of the image and audio may be reduced. The concatenated bytes may be then processed by a cross-attention mechanism to condense the concatenated bytes into a vector of a fixed size. The vectors are then used to train an ML model primarily using supervised approaches although self-supervised or unsupervised approaches may also be used.

In a second multimodal ML approach, data from different modalities may be kept separate at the input stage and used as inputs to different, modality-specific ML models (e.g., a CNN for image data and a recurrent neural network (RNN) for sequential data). The modality-specific ML models may be trained jointly such that information from across different modalities is combined to make predictions, and the combined (cross-modality) loss is used to adjust model weights. Alternatively, the modality-specific ML models may also be trained separately using a separate loss function for each modality. A combined ML model is then trained based on the outputs of the modality specific models. Continuing with the example, the training data for each modality-specific ML model may be based on its data along with a label. The combined ML model is then trained with the outputs of the modality-specific ML models with a final label.

Transformer ML employs an attention mechanism to process tokens representing the input in parallel.

A transformer ML model includes encoders with a neural network and self-attention mechanism and decoders with an attention mechanism, a self-attention mechanism, and a neural network.

The first layer of the encoder inputs the tokens, and each other layer inputs the encodings of the previous layer. The self-attention mechanism inputs the encodings of the previous layer (or tokens for the first encoder) and weighs the relevance of each encoding to each other to generate input for the neural network which generates the encodings.

The first layer of decoder inputs data based on the tokens, and each other layer inputs the decoding of the prior layer. Each layer of the decoder also inputs the encodings of the encoder. The attention and self-attention mechanisms input the decodings (or tokens for the first layer) and weigh the relevance of each decoding to each other to generate input for the neural network.

The AAML system may employ a transformer ML model. Transformer ML was introduced as an alternative to a recurrent neural network that is both more effective and more parallelizable. (See, Vaswani, A., Shazeer, N., Parmar, N., Uszkoreit, J., Jones, L., Gomez, A. N., Kaiser, Ł. and Polosukhin, I., 2017. Attention is all you need. Advances in neural information processing systems, 30, which is hereby incorporated by reference.) Transformer machine learning was originally described in the context of natural language processing (NLP) but has been adapted to other applications such as image processing to augment or replace a CNN. In the following, transformer machine learning is described in the context of NLP as introduced by Vaswani.

A transformer ML model includes an encoder whose output is input to a decoder. The encoder includes an input embedding layer followed by one or more encoder attention layers. The input embedding layer generates an embedding of the inputs. For example, if a transformer ML model is used to process an EHR, a feature that is text (e.g., medical provider comments) may be represented as tokens that each includes an embedding of a word and its positional information in the text. Other features may also be represented as tokens such as tokens corresponding to portions of a voltage-time series of an ECG or tokens corresponding to embedding of a treatment and time of treatment.

The first encoder attention layer inputs the embeddings and the other encoder attention layers input the output from the prior encoder attention layer. An encoder attention layer includes a multi-head attention mechanism followed by a normalization sublayer whose output is input to a feedforward neural network followed by a normalization sublayer. A multi-head attention mechanism includes multiple self-attention mechanisms that each inputs the encodings of the previous layer and weighs the relevance encodings to other encodings. For example, the relevance may be determined by the following attention function:

$$Attention ( Q , K , V ) = softmax ( \frac{{QK}^{T}}{\sqrt{{d}_{k}}} ) V$$

where Q represents a query, K represents a key, V represents a value, and dₖ represents the dimensionality of K. This attention function is referred to as scaled dot-product attention. In Vaswani, the query, key, and value of an encoder multi-head attention mechanism is set to the input of the encoder attention layer. The multi-head attention mechanism determines the multi-head attention as represented by the following:

$$MultiHead ( Q , K , V ) = concat ( {head}_{1} , … , {head}_{8} ) {W}^{o}$$

$${head}_{i} = Attention ( {QW}_{i}^{Q} , {KW}_{i}^{K} , {VW}_{i}^{V} )$$

where W represents weights that are learned during training. The weights for the feedforward networks are also learned during training. The weights may be initialized to random values. A normalization layer normalizes its input to a vector having a dimension as expected by the next layer or sub-layer.

The decoder includes an output embedding layer, decoder attention layers, a linear layer, and a softmax layer. The output embedding layer inputs the output of the decoder shifted right. Each decoder attention layer inputs the output of the prior decoder attention layer (or the output embedding layer) and the output of the encoder. The embedding layer is input to the decoder attention layer, the output of the decoder attention layer is input the linear layer, and the output of the linear layer is input to the softmax layer which outputs probabilities. A decoder attention layer includes a decoder masked multi-head attention mechanism followed by a normalization sublayer, a decoder multi-head attention mechanism followed by a normalization sublayer, and a feedforward neural network followed by a normalization sublayer. The decoder masked multi-head attention mechanism masks the input so that predictions for a position are only based on outputs for prior positions. A decoder multi-head attention mechanism inputs the normalized output of the decoder masked multi-head attention mechanism as a query and the output of the encoder as a key and a value. The feedforward neural network inputs the normalized output of the decoder multi-head attention mechanism. The normalized output of the feedforward neural network is the output of that multi-head attention layer. The weights of the linear layer are also learned during training.

The AAML system may employ fine-tuning, which is an ML technique to train an ML model by leveraging a previously trained ML model. For example, if an AF ML model has been trained to provide treatment assessments for an AF ablation, fine tuning may be used to train an PVI ML model to provide treatment assessments for a PVI ablation, which is a subcategory of an AF ablation. The inner layers of the PVI ML model have weights that are the same as the inner layers of the AF ML model. The input layer and the output layer of the PVI ML model are trained training feature vectors relating to a PVI. Thus, the training process needs only learn the weights for the input layer and the output layer. Alternatively, when training a PVI ML model, all the weights may be initialized to the weights of the AF ML model rather than to random values or default values (e.g., all zeros).

An unsupervised ML technique trains an ML model using unlabeled training data. K-means clustering is an example ML technique. Given feature vectors representing the training data, K-means clustering clusters the feature vectors into clusters of similar feature vectors. With K-means clustering, the number of clusters may be predefined. For example, the AAML system may employ 50 clusters (k=3) to represent clusters of training feature vectors derived from EHRs. An example training technique initially randomly places a training feature vector in each cluster. The training then repeatedly calculates a mean training feature vector of each cluster, selects a training feature vector not in a cluster, identifies the cluster whose mean is most similar, adds the training feature vector to that cluster, and moves the training feature vectors already in the clusters to the cluster with the most similar mean. Similarity may be determined, for example, based on Pearson correlation, cosine similarity, dot product, and so on. The training ends when all the training feature vectors have been added to a cluster. The AAML system derived the treatment option for a cluster from the treatments of the training feature vectors in the cluster. To identify a treatment option of a candidate, a candidate feature vector is generated, the cluster with a mean training feature vector that is most similar to that candidate feature vector is identified, and the treatment option of that cluster is output as the treatment option for the candidate.

The features employed by an ML model may also be latent vectors generated using a machine learning technique such as an autoencoder. For example, an autoencoder may be trained using ECG images. In such a case, when an ECG image is input into the trained autoencoder, the latent vector that is generated is a feature vector that represents the ECG image. That feature vector can be input into another trained ML model such as a neural network or support vector machine to generate an output. When training the other ML model, for example, to classify an ECG as representing an atrial fibrillation or a ventricular fibrillation, the training ECG images are input to the autoencoder to generate training feature vectors that are labeled as being atrial fibrillation or ventricular fibrillation. The other ML model is then trained using the labeled feature vectors. The autoencoder may be trained using the training ECG images or may have been previously trained using a collection of ECG images. Rather pre-training an autoencoder, only the portion of the autoencoder that generates the latent vector may be trained in parallel with the other ML model using a combined loss function. In such a case, no autoencoding is performed. Rather the latent vector represents features of an ECG image that are particularly relevant to generating the output of the other ML model. Such an ML architecture may be used, for example, when the other ML model (e.g., transformer) is not designed to process ECG images directly.

In some embodiments, a TOML model may be based on collaborative filtering. The AAML system may generate a patient-treatment matrix that identifies the treatments of each patient and possibly indicates the order of treatment. For example, the treatments for patients with an AF may be a PVI, SVC ablation, medication 1, medication 2, defer for 3 months, and defer for 6 months. The cells of the matrix may indicate the outcome of a treatment in the range of [−1.0, 1.0] with −1.0 being the most unfavorable and 1.0 being the most favorable. The patient-treatment matrix may be augmented with the patient features so that similarity between a candidate and the patients can influence the identification of a treatment option. The “recommended” treatment option may be identified using a weighted matrix factorization. A TAML model that provides a treatment assessment (e.g., risk) may be generated from the patient-treatment matrix. Rather than employing matrix factorization, logistic regression and a neural network may be used. The neural network (with a softmax layer at the end) may be trained using training data derived from the patient-treatment matrix. The patients included in the patient-treatment matrix may be identified based on similarity to the candidate using features derived from the EHRs such as demographics, current medical condition (e.g., AF and hypertension), and so on. The patients may be identified using, for example, K-means clustering. The treatment option for the candidate may be based on similarity between treatments and outcomes of the patients and past treatments and outcomes for candidate. A similarity score may be generated, for example, using Pearson correlation. If many of the most similar patients had a positive outcome for a treatment that the candidate did not have, the AAML system may recommend that treatment as a treatment option for the candidate.

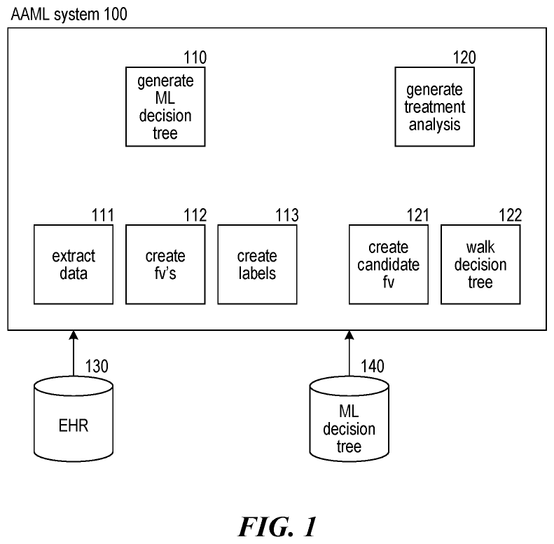

FIG. 1 is a block diagram that illustrates components of the AAML system in some embodiments. The AAML system 100 includes a generate ML decision tree component 110 and a generate assessment component 120. The generate ML decision tree component invokes an extract data component 111, a create feature vectors component 112, and a create labels component 113. The generate assessment component invokes a create candidate feature vector component 121 and a walk decision tree component 122. The AAML system accesses EHR data store 130 and ML decision tree data store 140.

The computing systems (e.g., network nodes or collections of network nodes) on which the AAML system and the other described systems may be implemented may include a central processing unit, input devices, output devices (e.g., display devices and speakers), storage devices (e.g., memory and disk drives), network interfaces, graphics processing units, communications links (e.g., Ethernet, Wi-Fi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, distributed systems, cloud-based computing systems, client computing systems that interact with cloud-based computing system, desktop computers, laptops, tablets, e-readers, personal digital assistants, smartphones, gaming devices, servers, and so on. The computing systems may access computer-readable media that include computer-readable storage mediums and data transmission mediums. The computer-readable storage mediums are tangible storage means that do not include a transitory, propagating signal. Examples of computer-readable storage mediums include memory such as primary memory, cache memory, and secondary memory (e.g., DVD), and other storage. The computer-readable storage media may have recorded on them or may be encoded with computer-executable instructions or logic that implements the AAML system and the other described systems. The data transmission media are used for transmitting data via transitory, propagating signals or carrier waves (e.g., electromagnetism) via a wired or wireless connection. The computing systems may include a secure crypto processor as part of a central processing unit (e.g., Intel Secure Guard Extension (SGX)) for generating and securely storing keys and for encrypting and decrypting data using the keys and for securely executing all or some of the computer-executable instructions of the AAML system. Some of the data sent by and received by the AAML system may be encrypted, for example, to preserve patient privacy (e.g., to comply with government regulations such the European General Data Protection Regulation (GDPR) or the Health Insurance Portability and Accountability Act (HIPAA) of the United States). The AAML system may employ asymmetric encryption (e.g., using private and public keys of the Rivest-Shamir-Adleman (RSA) standard) or symmetric encryption (e.g., using a symmetric key of the Advanced Encryption Standard (AES)).

The one or more computing systems may include client-side computing systems and cloud-based computing systems (e.g., public or private) that each executes computer-executable instructions of the AAML system. A client-side computing system may send data to and receive data from one or more servers of the cloud-based computing systems of one or more cloud data centers. For example, a client-side computing system may send a request to a cloud-based computing system to perform tasks such as generate a graph illustrating life expectancy by age or train a patient-specific AF ML model. A cloud-based computing system may respond to the request by sending to the client-side computing system data derived from performing the task such a graph. The servers may perform computationally expensive tasks in advance of processing by a client-side computing system such as training a patient-specific ML model using patient feature vectors that are similar to a candidate feature vector in response to request received from a client-side computing system. A client-side computing system may provide a user experience (e.g., user interface) to a user of the AAML system. The user experience may originate from a client computing device or a server computing device. For example, a client computing device may generate a patient-specific graphic of a heart and display the graphic. Alternatively, a cloud-based computing system may generate the graphic (e.g., in a Hyper-Text Markup Language (HTML) format or an extensible Markup Language (XML) format) and provide it to the client-side computing system for display. A client-side computing system may also send data to and receive data from various medical devices such as an ECG monitor, an ablation therapy device, an ablation planning device, and so on. The data received from the medical devices may include an ECG, actual ablation characteristics (e.g., ablation location and ablation pattern), and so on. The data sent to a medical device may include data, for example, data in a Digital Imaging and Communications in Medicine (DICOM) format. A client-side computing device may also send data to and receive data from medical computing systems that store EHRs, descriptions of medical devices (e.g., type, manufacturer, and model number) used in ablation procedures, and so on. The term cloud-based computing system may encompass computing systems of a public cloud data center provided by a cloud provider (e.g., Azure provided by Microsoft Corporation) or computing systems of a private server farm (e.g., operated by the provider of the AAML system).

The AAML system and the other described systems may be described in the general context of computer-executable instructions, such as program modules and components, executed by one or more computers, processors, or other devices. Generally, program modules or components include routines, programs, objects, data structures, and so on that perform tasks or implement data types of the AAML system and the other described systems. Typically, the functionality of the program modules may be combined or distributed as desired in various examples. Aspects of the AAML system and the other described systems may be implemented in hardware using, for example, an application-specific integrated circuit (ASIC) or a field programmable gate array (FPGA).

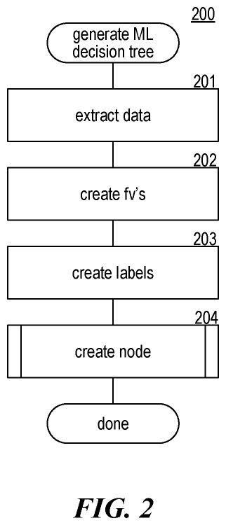

FIG. 2 is a flow diagram that illustrates the processing of a generate ML decision tree component of the AAML system in some embodiments. The generate ML decision tree component 200 generates the nodes of a decision tree based on patient data of the EHR data store and stores the ML decision tree in the ML decision tree data store. The component may be invoked to generate a TOML decision tree and a TAML decision tree. In block 201, the component invokes the extract data component to extract patient data to use in building the ML decision tree from the HR data store. In block 202, the component creates a feature vector for each patient based on the extracted patient data. In block 203, the component creates a label for each feature vector. The labels created when generating the TOML decision tree relate to patient treatments and when generating the TAML decision relate to patient outcomes. In block 204, the component invokes the create node component to create the nodes of the ML decision tree. When creating the treatment assessment nodes for the TAML decision tree, the component generates the treatment assessment based on the treatment outcomes based on the patient feature vectors that match the path from the root node to that treatment assessment node. The component then completes. Similar feature vectors may be employed when training non-decision tree ML models.

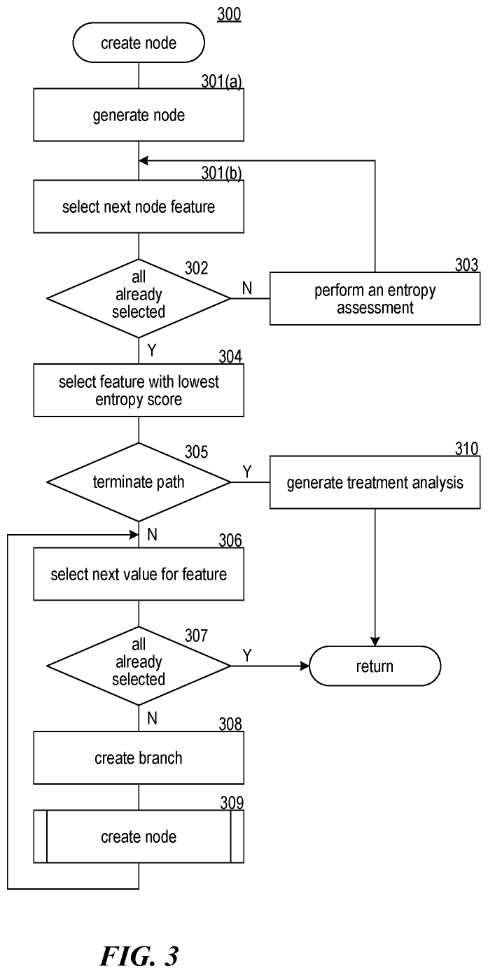

FIG. 3 is a flow diagram that illustrates the processing of a create node component of the AAML system in some embodiments. The create node component is initially invoked to create the root node and recursively invoked to create additional nodes of the ML decision tree. In block 301(a), the component creates a new node along the current path. In block 301(b), the component selects the next node feature out of the node features that are available for the newly created node. In decision block 302, if all the node features have already been selected, then the component continues at block 304, else the component continues at block 303. In block 303, the component generates an entropy score based on the selected node feature and loops to block 301 to select the next node feature. As discussed above, the entropy score is based on the distribution of the feature values for the selected node feature. In block 304, the component selects the node feature with the lowest entropy score. In decision block 305, if the current path satisfies a termination criterion, then the component continues at block 310, else the component continues at block 306. The termination criterion may be, for example, based on the entropy score satisfying an entropy score termination criterion. The entropy score termination criterion may be based on the entropy score being below a termination threshold. In block 306, the component selects the next feature value for the selected feature. The feature values are the feature values of the feature vectors that match the path to the created node. In decision block 307, if all the feature values have already been selected, then the component returns, else the component continues at block 308. In block 308, the component creates a branch for the selected feature value. In block 309, the component recursively invokes the create node component and then loops to block 306 to select the next value for the selected feature. In block 310, the component generates a treatment analysis based on whether the component is invoked to generate a TOML decision tree or a TAML decision tree. The component stores the treatment analysis in the created node and returns which terminates that path of the ML decision tree.

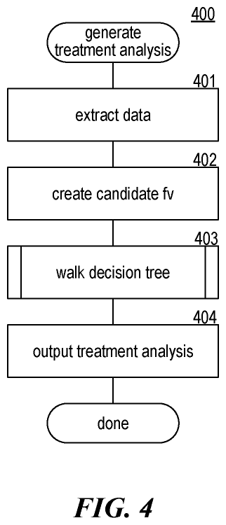

FIG. 4 is a flow diagram that illustrates the processing of a generate treatment analysis component of the AAML system in some embodiments. The treatment analysis component 400 generates a treatment option or a treatment assessment for a candidate. In block 401, the component extracts data relating to the candidate from the EHR data store. In block 402, the component creates a candidate feature vector. In block 403, the component invokes the walk decision tree component passing the candidate feature vector to walk a path of the TOML decision tree or TAML decision tree to identify treatment analysis. In block 404, the component outputs the treatment analysis and completes.

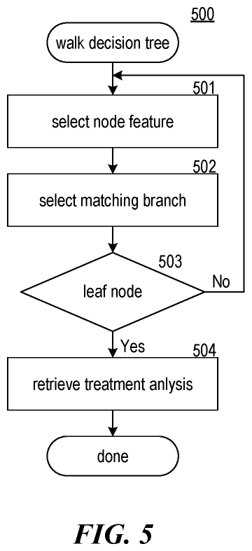

FIG. 5 is a flow diagram that illustrates the processing of a walk decision tree component of the AAML system in some embodiments. The walk decision tree component 500 is provided a candidate feature vector and walks a path of the TOML decision tree or the TAML decision tree to identify a treatment option or treatment assessment for the candidate. In block 501, the component selects the node feature of the current node starting with the root node. In block 502, the component identifies a matching branch that matches the feature value of the candidate feature for the selected node feature. In decision block 503, if the node at the end of the branch is a leaf node, then the component continues at block 504, else the component selects that node and loops to block 501 to select the node feature of that selected node to continue walking the path to a leaf node. In block 504, the component retrieves the treatment option or treatment analysis associated with the leaf node and then completes.

The following paragraphs describe various aspects of the AAML system. An implementation of the AAML system may employ any combination or sub-combination of the aspects and may employ additional aspects. The processing of the aspects may be performed by one or more computing systems with one or more processors that execute computer-executable instructions that implement the aspects and that are stored on one or more computer-readable storage mediums.

In some aspects, the techniques described herein relate to a method performed by one or more computing systems for providing a treatment analysis for a candidate for treatment of an arrhythmia, the method including: accessing an electronic health record data of the candidate; accessing a machine learning (ML) decision tree that includes decision nodes that each have one or more branches, each decision node corresponding to a feature, each branch of a decision node corresponding to a feature value of the feature of that decision node, wherein each leaf node corresponds to a treatment analysis based on feature values of a path from the root node to that leaf node; for each decision node along a path from the root node to a leaf node, selecting the feature of that decision node; retrieving a candidate feature value of the candidate for that feature; and selecting the node at the end of the branch of that decision node with a branch value that matches that candidate feature value; and outputting the treatment analysis associated with the leaf node. In some aspects, the techniques described herein relate to a method wherein a feature is arrhythmia type and further including selecting the ML decision tree based on the arrhythmia type of the candidate. In some aspects, the techniques described herein relate to a method further including generating the ML decision tree based on electronic health records of patients. In some aspects, the techniques described herein relate to a method wherein a feature is arrhythmia type and a candidate feature value for the arrhythmia type is determined dynamically based on electrocardiogram collected from the candidate. In some aspects, the techniques described herein relate to a method wherein a feature is source location of an arrhythmia and a candidate feature value for source location is determined dynamically based on electrocardiogram collected from the candidate when the feature is source location. In some aspects, the techniques described herein relate to a method wherein the treatment analysis is a treatment option. In some aspects, the techniques described herein relate to a method wherein the treatment analysis is a treatment assessment. In some aspects, the techniques described herein relate to a method wherein the treatment analysis is a combination of a treatment option and treatment assessment.

In some aspects, the techniques described herein relate to one or more computing systems for generating a machine learning (ML) decision tree for providing treatment analyses for a treatment relating to a medical condition, the one or more computing systems including: one or more computer-readable storage mediums that store computer-executable instructions for controlling the one or more computing systems to: access electronic health records of patients; for each patient treated for the medical condition, generate a patient feature vector based on feature values of features of the patient feature vector, the feature values derived from the electronic health record of that patient, at least one of the features corresponding to a treatment analysis of the treatment for that patient; generate the ML decision tree that includes decision nodes, treatment analysis nodes, and branches from decision nodes to other nodes, at least some of the decision nodes corresponding to a feature that is selected based on an entropy analysis of feature values of patient feature vectors, a branch from a decision node having a feature value of the feature of that decision node, the entropy analysis for a decision node based on patient feature vectors with feature values that match the branch values along the path from the root node to that decision node; for each treatment analysis node, generate a treatment analysis based on treatments of patient feature vectors with feature values the match branch values along the path from the root node to that treatment analysis node; and one or more processors for controlling the one or more computing systems to execute one or more of the computer-executable instructions. In some aspects, the techniques described herein relate to one or more computing systems wherein the ML decision tree is based on multiple treatment options with subtrees corresponding to each treatment option. In some aspects, the techniques described herein relate to one or more computing systems wherein a treatment analysis is a treatment assessment that relates chance of the treatment being successful, recurrence of the medical condition, and/or lifespan extension. In some aspects, the techniques described herein relate to one or more computing systems wherein the treatment assessment of a treatment analysis node is generated based on treatment outcomes of patient feature vectors with feature values along the path from the root node to that treatment analysis node. In some aspects, the techniques described herein relate to one or more computing systems wherein the computer-executable instructions further include instructions to receive feature values of a candidate feature vector of a candidate for the treatment, identify a path of the ML decision tree from the root node to a treatment analysis node based on the feature values, and output an indication of the treatment analysis of that treatment analysis node. In some aspects, the techniques described herein relate to one or more computing systems wherein the medical condition is an arrhythmia and a treatment analysis is a treatment option that is a cardiac ablation, arrhythmia medication, and/or take no action. In some aspects, the techniques described herein relate to one or more computing systems wherein the treatment analysis is a treatment option. In some aspects, the techniques described herein relate to one or more computing systems wherein the treatment analysis is a treatment assessment. In some aspects, the techniques described herein relate to one or more computing systems wherein the treatment analysis is a combination of a treatment option and treatment assessment.

In some aspects, the techniques described herein relate to one or more computing systems for providing a treatment analysis for a candidate for treatment of an arrhythmia, the one or more computing systems including: one or more computer-readable storage mediums that store computer-executable instructions for controlling the one or more computing systems to: access a candidate feature vector with feature values for features of the candidate; identify a path from a root node to a leaf node of an arrhythmia machine learning (ML) decision tree based on the candidate feature vector, the arrhythmia ML decision tree including non-leaf nodes that each correspond to a feature and having one or more branches corresponding to feature values for that feature and including leaf nodes that each correspond to a treatment analysis based on feature values of a path from the root node to that leaf node; and outputting the treatment analysis associated with the leaf node of the identified path. In some aspects, the techniques described herein relate to one or more computing systems wherein the instructions further when a feature of a non-leaf node is a source location of an arrhythmia applying an ablation pattern system to a cardiogram of the candidate to identify a feature value that is a source location. In some aspects, the techniques described herein relate to one or more computing systems wherein the instructions further when a feature of a non-leaf node is an ablation pattern applying a mapping function to a cardiogram of the candidate to identify a feature value that is an ablation pattern. In some aspects, the techniques described herein relate to one or more computing systems wherein the instructions further when a feature of a non-leaf node is based on a catheter route for an ablation of an arrhythmia applying a route traversal system to a target location for the ablation and anatomical characteristics of the candidate. In some aspects, the techniques described herein relate to one or more computing systems further including instructions that when identifying a path, input from a user a feature value. In some aspects, the techniques described herein relate to one or more computing systems further including instructions that identify an arrhythmia type.

In some aspects, the techniques described herein relate to a method performed by one or more computing systems including: generating, from electronic health records of patients treated for an arrhythmia, patient feature vectors based on features having feature values; labeling each patient feature vector with a label generated based on an arrhythmia outcome of treatment of the arrhythmia; and training a machine learning (ML) model with training data that includes the patient feature vectors and labels. In some aspects, the techniques described herein relate to a method wherein the ML model is a decision tree. In some aspects, the techniques described herein relate to a method further including generating treatment assessments for treatment assessment nodes of the ML model based on the arrhythmia treatment outcomes. In some aspects, the techniques described herein relate to a method wherein the ML model is a recommender system. In some aspects, the techniques described herein relate to a method wherein the ML model is a neural network. In some aspects, the techniques described herein relate to a method wherein the ML model is a K-Nearest Neighbor model. In some aspects, the techniques described herein relate to a method wherein the ML model is based on K-means clustering. In some aspects, the techniques described herein relate to a method further including applying the trained ML model to a candidate feature vector of a candidate to identify an arrhythmia outcome for the candidate. In some aspects, the techniques described herein relate to a method wherein the generating includes deriving a feature value from one or more fields of an electronic health record. In some aspects, the techniques described herein relate to a method wherein the generating is based on running simulations of electrical activity of a heart to assess results of ablations targeting various target locations. In some aspects, the techniques described herein relate to a method wherein a feature relates to coronary calcium. In some aspects, the techniques described herein relate to a method wherein a feature vector is a latent vector derived from the feature values of the features.

In some aspects, the techniques described herein relate to a method performed by one or more computing systems to assist a medical provider during an ablation procedure for a candidate, the method including: prior to the ablation procedure, generating a candidate feature vector with features having feature values derived from an electronic health record of the candidate; and applying a machine learning (ML) model to the candidate feature vector to generate a treatment analysis relating to the ablation procedure; and during the ablation procedure, updating a feature value of a feature of the candidate feature vector based on a condition of the candidate that is determined during the ablation procedure; and applying the ML model to the updated candidate feature vector to generate an updated treatment analysis for the candidate. In some aspects, the techniques described herein relate to a method wherein the updated feature value relates to an ablation performed during the procedure. In some aspects, the techniques described herein relate to a method wherein the updated feature value relates to cardiac tissue characteristics. In some aspects, the techniques described herein relate to a method wherein the updated feature value relates to cardiac geometry. In some aspects, the techniques described herein relate to a method wherein the ML model is an ML decision tree.

All documents incorporated by reference are incorporated in their entirety for the full extent of their disclosures. In the event of inconsistencies between the language in this document and any incorporated-by-reference document, the language in the incorporated-by-reference document should be considered supplementary to that of this document and the language in this document controls.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

I/We claim:

1. A method performed by one or more computing systems for providing a treatment analysis for a candidate for treatment of an arrhythmia, the method comprising:
accessing an electronic health record data of the candidate;
accessing a machine learning (ML) decision tree that includes decision nodes that each have one or more branches, each decision node corresponding to a feature, each branch of a decision node corresponding to a feature value of the feature of that decision node, wherein each leaf node corresponds to a treatment analysis based on feature values of a path from the root node to that leaf node;
for each decision node along a path from the root node to a leaf node, selecting the feature of that decision node;
retrieving a candidate feature value of the candidate for that feature; and
selecting the node at the end of the branch of that decision node with a branch value that matches that candidate feature value; and
outputting the treatment analysis associated with the leaf node.

2. The method of claim 1 wherein a feature is arrhythmia type and further comprising selecting the ML decision tree based on the arrhythmia type of the candidate.

3. The method of claim 1 further comprising generating the ML decision tree based on electronic health records of patients.

4. The method of claim 1 wherein a feature is arrhythmia type and a candidate feature value for the arrhythmia type is determined dynamically based on electrocardiogram collected from the candidate.

5. The method of claim 1 wherein a feature is source location of an arrhythmia and a candidate feature value for source location is determined dynamically based on electrocardiogram collected from the candidate when the feature is source location.

6. The method of claim 1 wherein the treatment analysis is a treatment option.

7. The method of claim 1 wherein the treatment analysis is a treatment assessment.

8. The method of claim 1 wherein the treatment analysis is a combination of a treatment option and treatment assessment.

9. One or more computing systems for generating a machine learning (ML) decision tree for providing treatment analyses for a treatment relating to a medical condition, the one or more computing systems comprising:
one or more computer-readable storage mediums that store computer-executable instructions for controlling the one or more computing systems to:
access electronic health records of patients;
for each patient treated for the medical condition, generate a patient feature vector based on feature values of features of the patient feature vector, the feature values derived from the electronic health record of that patient, at least one of the features corresponding to a treatment analysis of the treatment for that patient;
generate the ML decision tree that includes decision nodes, treatment analysis nodes, and branches from decision nodes to other nodes, at least some of the decision nodes corresponding to a feature that is selected based on an entropy analysis of feature values of patient feature vectors, a branch from a decision node having a feature value of the feature of that decision node, the entropy analysis for a decision node based on patient feature vectors with feature values that match the branch values along the path from the root node to that decision node;
for each treatment analysis node, generate a treatment analysis based on treatments of patient feature vectors with feature values the match branch values along the path from the root node to that treatment analysis node; and
one or more processors for controlling the one or more computing systems to execute one or more of the computer-executable instructions.

10. The one or more computing systems of claim 9 wherein the ML decision tree is based on multiple treatment options with subtrees corresponding to each treatment option.

11. The one or more computing systems of claim 9 wherein a treatment analysis is a treatment assessment that relates chance of the treatment being successful, recurrence of the medical condition, and/or lifespan extension.

12. The one or more computing systems of claim 11 wherein the treatment assessment of a treatment analysis node is generated based on treatment outcomes of patient feature vectors with feature values along the path from the root node to that treatment analysis node.

13. The one or more computing systems of claim 9 wherein the computer-executable instructions further include instructions to receive feature values of a candidate feature vector of a candidate for the treatment, identify a path of the ML decision tree from the root node to a treatment analysis node based on the feature values, and output an indication of the treatment analysis of that treatment analysis node.

14. The one or more computing systems of claim 9 wherein the medical condition is an arrhythmia and a treatment analysis is a treatment option that is a cardiac ablation, arrhythmia medication, and/or take no action.

15. The one or more computing systems of claim 9 wherein the treatment analysis is a treatment option.

16. The one or more computing systems of claim 9 wherein the treatment analysis is a treatment assessment.

17. The one or more computing systems of claim 9 wherein the treatment analysis is a combination of a treatment option and treatment assessment.

18. One or more computing systems for providing a treatment analysis for a candidate for treatment of an arrhythmia, the one or more computing systems comprising:
one or more computer-readable storage mediums that store computer-executable instructions for controlling the one or more computing systems to:
access a candidate feature vector with feature values for features of the candidate;
identify a path from a root node to a leaf node of an arrhythmia machine learning (ML) decision tree based on the candidate feature vector, the arrhythmia ML decision tree including non-leaf nodes that each correspond to a feature and having one or more branches corresponding to feature values for that feature and including leaf nodes that each correspond to a treatment analysis based on feature values of a path from the root node to that leaf node; and
outputting the treatment analysis associated with the leaf node of the identified path.

19. The one or more computing systems of claim 18 wherein the instructions further when a feature of a non-leaf node is a source location of an arrhythmia applying an ablation pattern system to a cardiogram of the candidate to identify a feature value that is a source location.

20. The one or more computing systems of claim 18 wherein the instructions further when a feature of a non-leaf node is an ablation pattern applying a mapping function to a cardiogram of the candidate to identify a feature value that is an ablation pattern.

21. The one or more computing systems of claim 18 wherein the instructions further when a feature of a non-leaf node is based on a catheter route for an ablation of an arrhythmia applying a route traversal system to a target location for the ablation and anatomical characteristics of the candidate.

22. The one or more computing systems of claim 18 further comprising instructions that when identifying a path, input from a user a feature value.

23. The one or more computing systems of claim 18 further comprising instructions that identify an arrhythmia type.

24. A method performed by one or more computing systems comprising:
generating, from electronic health records of patients treated for an arrhythmia, patient feature vectors based on features having feature values;
labeling each patient feature vector with a label generated based on an arrhythmia outcome of treatment of the arrhythmia; and
training a machine learning (ML) model with training data that includes the patient feature vectors and labels.

25. The method of claim 24 wherein the ML model is a decision tree.

26. The method of claim 25 further comprising generating treatment assessments for treatment assessment nodes of the ML model based on the arrhythmia treatment outcomes.

27. The method of claim 24 wherein the ML model is a recommender system.

28. The method of claim 24 wherein the ML model is a neural network.

29. The method of claim 24 wherein the ML model is a K-Nearest Neighbor model.

30. The method of claim 24 wherein the ML model is based on K-means clustering.

31. The method of claim 24 further comprising applying the trained ML model to a candidate feature vector of a candidate to identify an arrhythmia outcome for the candidate.

32. The method of claim 24 wherein the generating includes deriving a feature value from one or more fields of an electronic health record.

33. The method of claim 24 wherein the generating is based on running simulations of electrical activity of a heart to assess results of ablations targeting various target locations.

34. The method of claim 24 wherein a feature relates to coronary calcium.

35. The method of claim 24 wherein a feature vector is a latent vector derived from the feature values of the features.

36. A method performed by one or more computing systems to assist a medical provider during an ablation procedure for a candidate, the method comprising:
prior to the ablation procedure,
generating a candidate feature vector with features having feature values derived from an electronic health record of the candidate; and
applying a machine learning (ML) model to the candidate feature vector to generate a treatment analysis relating to the ablation procedure; and
during the ablation procedure,
updating a feature value of a feature of the candidate feature vector based on a condition of the candidate that is determined during the ablation procedure; and
applying the ML model to the updated candidate feature vector to generate an updated treatment analysis for the candidate.

37. The method of claim 36 wherein the updated feature value relates to an ablation performed during the procedure.

38. The method of claim 37 wherein the updated feature value relates to cardiac tissue characteristics.

39. The method of claim 37 wherein the updated feature value relates to cardiac geometry.

40. The method of claim 37 wherein the ML model is an ML decision tree.
